(19) United States
(12) Patent Application Publication
(10) Pub. No.: US 2007/0179376 A1
(43) Pub. Date: Aug. 2, 2007

(54) ELECTRODE ARRAY AND SYSTEM FOR DETECTING SIGNALS FOR ELECTROCARDIOGRAMS

(75) Inventor: Henning Gerder, Luebeck (DE)

Correspondence Address: MCGLEW & TUTTLE, PC, P.O. BOX 9227, SCARBOROUGH STATION, SCARBOROUGH, NY, 10510-9227, US

(73) Assignee: DRAEGER MEDICAL AG & CO. KG, Luebeck (DE)

(21) Appl. No.: 11/567,482

(22) Filed: Dec. 6, 2006

(30) Foreign Application Priority Data
Feb. 2, 2006 (DE) 10 2006 004 683.8

(51) Int. Cl. A61B 5/04

(52) U.S. Cl. 600/393; 600/509; 128/903

(57) ABSTRACT

An electrode array is provided for a system for recording electrocardiogram signals. The array includes a sheet-like holding element, with a first electrode, a second electrode and a third electrode for connection to the skin of a patient. The electrodes are arranged at the holding element. A transmitting device is arranged at the holding element. A system is also provided for detecting electrocardiogram signals that includes the electrode array. The electrode array as well as the system for detecting electrocardiogram signals fastens a plurality of electrodes on the body of a patient without these having to be connected to one another based on the provision of a reference potential terminal (6). The reference potential terminal (6) is connected to the first electrode (3) and to the second electrode (4) and makes available a reference potential. The reference potential terminal (6) and the third electrode (5) are connected to the transmitting device.

### CROSS REFERENCE TO RELATED APPLICATIONS

This application claims the benefit of priority under 35 U.S.C. §119 of German Patent Application DE 10 2006 004 683.8 filed Feb. 2, 2006, the entire contents of which are incorporated herein by reference.

### FIELD OF THE INVENTION

The present invention pertains to an electrode array for a system for recording electrocardiogram signals with a sheet-like holding element, with a first electrode, with a second electrode and with a third electrode for connection to the skin of a patient, the electrodes being arranged at the holding element, and with a transmitting means arranged at the holding element. Furthermore, the present invention pertains to a system for detecting electrocardiogram signals.

### BACKGROUND OF THE INVENTION

The electrocardiogram has proved to an indispensable diagnostic tool for monitoring the function of the heart as well as for diagnosing dysfunctions of the heart. This diagnostic method is based on the electric potentials of the skin of a patient, which are elicited by the electric stimulation of the heart muscle. These electric potentials are measured relative to a reference potential during the detection of an electrocardiogram. For evaluation, the differences of the potentials between individual electrodes located apart from each other in space are formed and plotted as a function of time. These so-called leads represent vectors of the stimulus propagation originating from the heart muscle as a function of time, so that it is possible in this manner to monitor the activity of the heart muscle.

To record an electrocardiogram, it is necessary to arrange a large number of electrodes distributed over the patient's skin over the entire body and to connect these to an evaluating unit. The problem arising first in this connection is that cable connections are necessary between the electrodes and the evaluating unit, and these represent a hindrance to the patient. In addition, these connections are extraordinarily disturbing, especially if the patient has to be transported.

A system for recording electrocardiogram signals, which comprises a plurality of electrodes, which are arranged at an elastic connection means and are all electrically connected to a transmitting means, which is likewise arranged at the connection means, is known for this from U.S. Pat. No. 6,611,705. The signals detected at the electrodes are transmitted to the evaluating unit in a wireless manner by means of the transmitting means, so that the cable connections between the patient and the evaluating unit are evaluated. However, the problem arises that the signals are measured in relation to a reference potential, which must likewise be made available in the system. This in turn requires that all electrodes of the system must be electrically connected to one another, so that the connection means must extend over great distances over the patient's body, which is uncomfortable for the patient. In addition, this makes it difficult to put on the system.

A system for recording electrocardiogram signals with a plurality of electrodes, which are arranged at an elastic fastening element, is known from WO 94/01039. Furthermore, the system has an evaluating and transmitting means, which is arranged at the fastening element and directly determines the needed leads from the signals detected relative to a common reference potential. An electrode used to detect the reference potential is additionally arranged here at the fastening element. Instead of the signals detected directly at the electrodes, the leads are then transmitted to a receiving means in a wireless manner.

It is necessary in this system as well that all electrodes must be connected to one another in order to make it possible to detect the signals relative to the reference potential. It is therefore comparatively complicated to put on this system as well. This is especially true if it is necessary to place electrodes not only in the area of the chest of the patient, but also at the extremities.

### SUMMARY OF THE INVENTION

Based on the state of the art, the basic object of the present invention is therefore to provide an electrode array as well as a system for detecting electrocardiogram signals, with which it is possible to fasten a plurality of electrodes on the body of a patient for recording an electrocardiogram without having to connect the electrodes to one another.

This object is accomplished according to the present invention by a first reference potential terminal being provided, which is connected to the first electrode and to the second electrode, and at which a reference potential is made available, and by the first reference potential terminal and the third electrode being connected to the transmitting means.

Instead of a plurality of electrodes connected to one another, which are arranged at a common holding element, a plurality of electrode arrays according to the present invention are arranged on the body of a patient for recording an electrocardiogram. The electric potential on the surface of the patient's skin is detected now in such a way that the potential of the third electrode is measured in relation to the reference potential made available at the first reference potential terminal. Since both the reference potential terminal and the third electrode are connected to the transmitting means, this difference signal can subsequently be transmitted to a receiving and evaluating means in a wireless manner. The leads can then be formed in the receiving and evaluating means by forming the difference between the signals of different electrode arrays.

Due to the reference potential made available in the electrode array, it becomes unnecessary to electrically connect the individual electrode arrays to one another. The reference potential necessary for the measurement is obtained, instead, as a virtual center from the potential difference between two electrodes of a three-electrode array. The reference potential thus obtained is similar over the entire body and therefore represents a common reference potential, to which the individual signals measured at the third electrode of an electrode array are related, so that these signals can then also be transmitted individually to a receiving and evaluating unit in a wireless manner.

It thus becomes easier to put on the electrode arrays according to the present invention, because they can also be fastened independently from one another, for example, also on the extremities of a patient.

In a preferred embodiment, a first resistor is provided between the first reference potential terminal and the first electrode and a second resistor is provided between the first reference potential terminal and the second electrode. A voltage divider is formed in this manner between the first electrode and the second electrode in order to thus make available the desired reference potential by forming the difference.

In another preferred manner, the first resistor and the second resistor are adjustable. This may also be embodied in such a way that switchable drain-source paths from field effect transistors (FETs) or bipolar transistors are used as an adjustable resistor. Besides the adjustable resistors, it is also possible that additional semiconductor elements, such as diodes, are used to build up a network, by means of which the reference potential can be adapted.

In addition, a first capacitor and/or a first inductance may be connected between the first reference potential terminal and the first electrode, and a second capacitor and/or a second inductance may be connected between the first reference potential terminal and the second electrode. A complex resistor can be embodied in this manner between the electrodes and the respective reference potential terminal in order to take into account not only a purely resistive but also a capacitive coupling of the electrodes to the patient's skin.

In another preferred manner, the first reference potential terminal may also be connected to the third electrode, in which case a third resistor is provided between the first reference potential terminal and the third electrode. The electrodes are connected to the first reference potential terminal in a star-shaped pattern in this case, so that the reference potential represents the center between the potentials of the three electrodes of the electrode array, which leads to improved comparability of the individual signals detected at different electrode arrays. Connecting this reference potential terminal to the ground terminal of a battery supply unit of an amplifier in the electrode array brings about, via the capacity of the battery and the time constant resulting therefrom, the suppression of interference signals and a temporarily static difference formation of the potentials at the amplifier input.

Furthermore, the first electrode and the second electrode are preferably connected to the transmitting means, so that, on the whole, an impedance network is formed. This makes it possible to detect a total of three individual signals on an electrode array by forming the difference with the reference potential, so that the leads can later be determined from a larger number of individual signals, which leads to improved accuracy.

It is, furthermore, preferred for the detection of the individual signals if a first signal amplifier, a second signal amplifier and a third signal amplifier are provided. Instrument amplifiers or chopper amplifiers are especially preferably used, and drift effects are avoided by the latter. The input of the first signal amplifier is connected to the first reference potential terminal and to the third electrode, the input of the second signal amplifier to the first reference potential terminal and to the first electrode and the input of the third signal amplifier to the first reference potential terminal and to the second electrode. Finally, the outputs of the signal amplifiers are connected to the transmitting means.

In such an array, the signals to be transmitted via the transmitting means are determined and amplified in a simple manner by forming the difference, the signals originating from the signal amplifiers being then related to the ground potential of the power supply unit (battery) of the amplifier stage of this array or to another reference potential level generated, which is related to the power supply.

In an alternative to the star connection of the first reference potential terminal with the electrodes, a second reference potential terminal may be provided, which is connected to the second electrode and to the third electrode. Furthermore, a third reference potential terminal may be provided, which is connected to the first electrode and the third electrode, the second reference potential terminal and the third reference potential terminal as well as the second electrode and the third electrode being connected to the transmitting means. In another preferred manner, a third resistor is provided between the second reference potential terminal and the second electrode, and a fourth resistor is provided between the second reference potential terminal and the third electrode. In addition, a fifth resistor is provided between the third reference potential terminal and the third electrode and a sixth resistor is provided between the third reference potential terminal and the first electrode.

As in the star connection, the resistors may be adjustable, on the one hand, in the manner already described, and capacitors and inductances may also be provided, besides ohmic resistors, in order to embody complex resistors or a complex network.

In this case of a delta connection of the three electrodes, a plurality of reference potentials are made available in an electrode array instead of only one reference potential, and a respective other reference potential can be used to detect an individual signal. The individual signals can therefore be detected independently from one another, so that a system of a plurality of electrode arrays is less error-prone.

A first signal amplifier, a second signal amplifier and a third signal amplifier are provided in another preferred manner. The input of the first signal amplifier is connected to the first reference potential terminal and to the third electrode, the input of the second signal amplifier to the second reference potential terminal and to the first electrode, and the input of the third signal amplifier to the third reference potential terminal and to the second electrode. Furthermore, the outputs of the signal amplifiers are connected to the transmitting means in this embodiment, so that the signals to be transmitted can be determined by forming the difference in a simple manner in this case as well, the signals sent being likewise related to the ground potential of the power supply unit.

In another preferred embodiment, a fourth electrode is provided, which is fastened to the holding element and is connected to the transmitting means. As a result, a fourth individual signal can also be detected, besides the three individual signals that are detected at the first, second and third electrodes, which are connected at the same time to a reference potential terminal. In another preferred manner, a fourth signal amplifier is provided, and the fourth electrode and the first reference potential terminal are connected to the inputs of the fourth signal amplifier and the output of the fourth signal amplifier is connected to the transmitting means. The fourth electrode can operate with its amplifier stage directly against the earth pole of the power supply unit (battery) or via a complex resistor network. This ensures active shielding.

The fourth electrode may also be connected to the ground of the supply unit directly (without a fourth amplifier stage) or indirectly via a complex resistor network as a passive shielding. However, it is also possible to connect the fourth electrode against the reference center of the star array via a differential amplifier. The difference thus formed contains signal components which contain common-mode components as well as static and dynamic asymmetries of build-up and contacting. These signal components can be used to correct the signal curves if this is permitted by the signal quality of the electrode signals.

To achieve good shielding effect, the fourth electrode may have a ring-shaped design, in which case it surrounds the first, second and third electrodes.

The holding element preferably has an upper part and a lower part, the electrodes being fastened to the lower part and the transmitting means being provided on the upper part. This makes possible simple assembly, cleaning of the electrodes as well as simple manufacture of the electrode array. In addition, the separation into a multilayer system offers advantages in case of battery replacement.

Furthermore, the holding element is preferably additionally provided with a power supply unit to supply the electrode array. For example, a foil battery may be used as a power supply unit for the electrode array, and it can then be easily replaced because of the splitting of the electrode array.

Finally, to make it possible to transmit the individual signals in a wireless manner between the electrode arrays, the electrode arrays have a receiving means in a preferred embodiment.

Furthermore, the above object is accomplished by the system for receiving electrocardiogram signals with a plurality of electrode arrays and with a receiving and evaluating means, wherein the electrode arrays have a sheet-like holding element, a first electrode, a second electrode and a third electrode for connection to the skin of a patient and a transmitting means arranged at the holding element, the electrodes being arranged at the holding element, and a first reference potential terminal being provided, which is connected to the first electrode and to the second electrode and at which a reference potential is made available, and the first reference potential terminal and the third electrode being connected to the transmitting means.

The system according to the present invention has the advantage that the need to connect the individual electrode arrays to one another via a cable connection is eliminated due to the reference potential made available at the electrode arrays. The system according to the present invention can therefore be put on more easily and is more pleasant for the patient to wear.

In a preferred embodiment of the system, the system has, furthermore, a transmission electrode array, which has a sheet-like holding element, a first electrode, a second electrode and a third electrode for connection to the skin of a patient, a transmission unit arranged at the holding element and a receiving unit arranged at the holding element, the electrodes being arranged at the holding element, and a first reference potential terminal being provided, which is connected to the first electrode and to the second electrode and at which a reference potential is made available, the first reference potential terminal, the third electrode and the receiving unit being connected to the transmission unit, the receiving unit being designed to receive signals of the transmitting means of the electrode arrays, and the transmitting unit being designed to transmit data to the receiving and evaluating means.

In this embodiment, the individual signals detected at the electrode arrays can first be sent in a wireless manner to the transmission electrode array, from which they are then transmitted to the receiving and evaluating unit by means of the transmission unit. A first evaluation may already take place in the transmission electrode array.

In a first alternative, the transmitting means is connected to the receiving and evaluating unit via a wireless connection. A staggered radio connection is embodied in this manner between the electrode arrays and the receiving and evaluating unit. This is associated with the advantage that the transmission power to be produced at the electrode arrays is low, because the signal needs only to be sent from these to the transmission electrode array, which is likewise arranged on the patient's body. The individual signals of the electrode arrays are received there by the receiving unit and sent together to the receiving and evaluating unit by means of the transmission unit. A high transmission power is necessary only during the joint transmission, so that increased energy consumption occurs only at the transmission electrode array.

The transmission unit may have a detachably arranged transmitter, and the transmission unit may, furthermore, be connected to the receiving and evaluating unit via a cable connection. Either the wireless connection or a cable is used between the patient and the receiving and evaluating unit in this case. In case of a wireless connection, the transmission unit and the receiving and evaluating unit may also be connected to one another via optical interfaces, for example, IrDA.

If a patient is treated as an inpatient, the cable connection may be used, whereas a wireless connection is used via the transmitter between the receiving and evaluating unit in case of a “mobile” patient.

The present invention will be explained below on the basis of exemplary embodiments, which are only preferred exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of the lower part of a first exemplary embodiment of an electrode array;

FIG. 2 is a schematic view of the upper part of the first exemplary embodiment of the electrode array;

FIG. 3 is a view of an alternative embodiment of the electrodes at the lower part for the first exemplary embodiment;

FIG. 4 is a schematic view of a second exemplary embodiment of an electrode array according to the present invention;

FIG. 5 is a schematic view of a third exemplary embodiment of an electrode array according to the present invention;

FIG. 6 is a schematic view of a first embodiment of a system according to the present invention for detecting an electrocardiogram;

FIG. 7 is a schematic view of a second embodiment of a system according to the present invention; and

FIG. 8 is a schematic top view of the upper part of a transmission electrode array.

### DESCRIPTION OF THE PREFERRED EMBODIMENTS

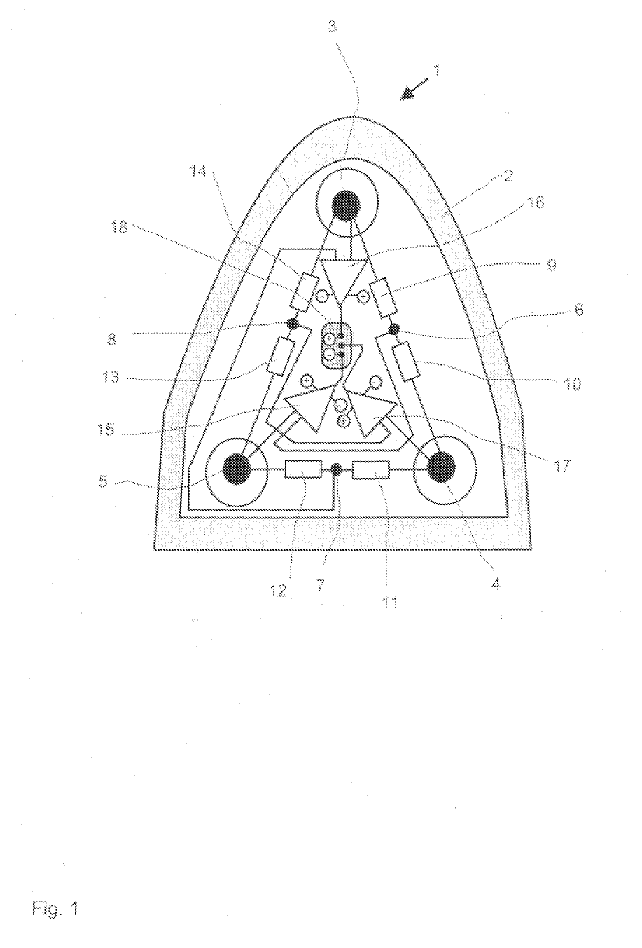

Referring to the drawings in particular, FIG. 1 shows an electrode array 1 according to the present invention, specifically the lower part 2. A first electrode 3, a second electrode 4 and a third electrode 5, which may be provided with a ring made of a contact gel, are arranged in the lower part 2 of an elastic, sheet-like holding element. The electrodes 3, 4 and 5 are fastened to the lower part 2 of the holding element such that they are in contact with the skin of a patient when the lower part 2 is attached to the skin.

Furthermore, the lower part 2 of the holding element has a first reference potential terminal 6, a second reference potential terminal 7 and a third reference potential terminal 8. The first reference potential terminal 6 is connected to both the first electrode 3 and the second electrode 4, a first resistor 9 being arranged between the first reference potential terminal 6 and the first electrode 3 and a second resistor 10 being arranged between the first reference potential terminal 6 and the second electrode 4. The second reference potential terminal 7 is connected in the same manner to the second electrode 4 and to the fourth electrode 5 via a third resistor 11 and a fourth resistor 12. Finally, the third reference potential terminal 8 is also connected to the first electrode 3 and to the third electrode 5 via a fifth resistor 13 and a sixth resistor 14.

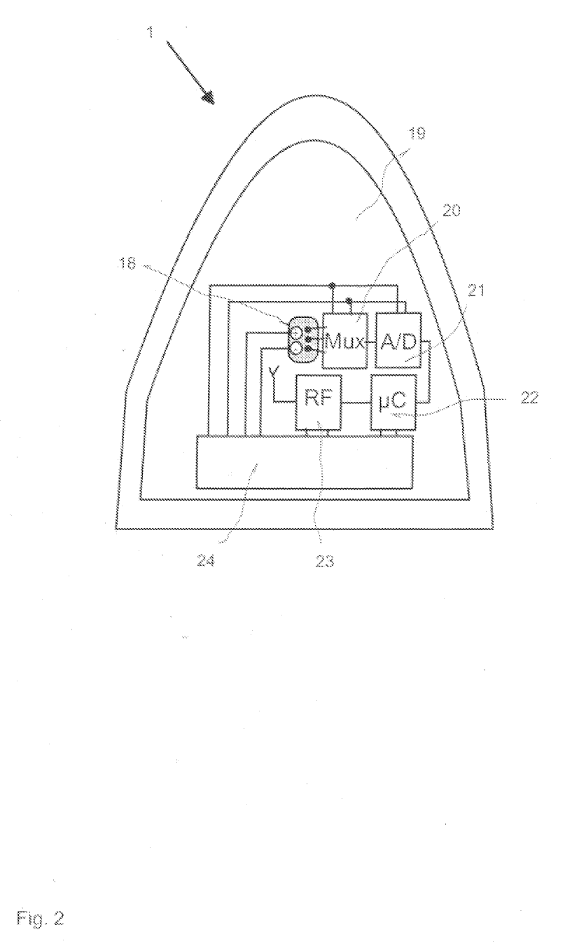

Furthermore, a first signal amplifier 15, a second signal amplifier 16 and a third signal amplifier 17 are provided on the lower part 2 of the holding element of the electrode array 1. The input of the first signal amplifier 15 is connected to the third electrode 5 and to the first reference potential terminal 6. Analogously, the second signal amplifier 16 is connected to the second reference potential terminal 7 and to the first electrode 3, and the third signal amplifier 17 is connected to the second electrode 4 and to the third reference potential terminal 8. The outputs of the signal amplifiers 15, 16 and 17 are connected to the input of a plug connection 18, which connects the lower part 2 of the electrode array 1 to the upper part 19 shown in FIG. 2.

It is also conceivable that the resistors 9, 10, 11, 12, 13 and 14 are adjustable, which can be embodied in such a way that switchable drain-source paths from field effect transistors (FETs) or bipolar transistors are used as an adjustable resistor. Besides the adjustable resistors, it is also possible that additional semiconductor elements such as diodes are used to build up a complex network, by means of which the reference potential can be adapted. In addition, a capacitor (capacitance) and/or an inductor (inductance) each may be connected between the reference potential terminals 6, 7, 8 and the electrodes 3, 4, 5, so that a complex resistor can be embodied between the electrodes and the respective reference potential terminals in order to take into account not only a purely resistive coupling but also a capacitive coupling of the electrodes with the patient's skin.

The upper part 19 of the electrode array 1 has first the second part of the plug connection 18, by means of which the signal amplifiers 15, 16 and 17 arranged at the lower part 2 are connected to the components of the upper part 19. A multiplexing unit 20, an analog-digital converter 21, a controller 22 and a high-frequency transmission stage 23 with an antenna are arranged on the upper part 19. Furthermore, a power supply unit 24 is provided, which also supplies the signal amplifiers 15, 16 and 17, besides the units on the upper part 19. As an alternative, the power supply unit may also be arranged in a part (not shown) separated from the lower part and the upper part. The multiplexing unit 20, the analog-digital converter 21, the controller 22 and the high-frequency transmission stage 23 together form the transmitting means of the electrode array 1.

Three individual signals, which reflect the electric potential on the patient's skin, can be detected and transmitted with the electrode array 1 shown in FIGS. 1 and 2 in the following manner.

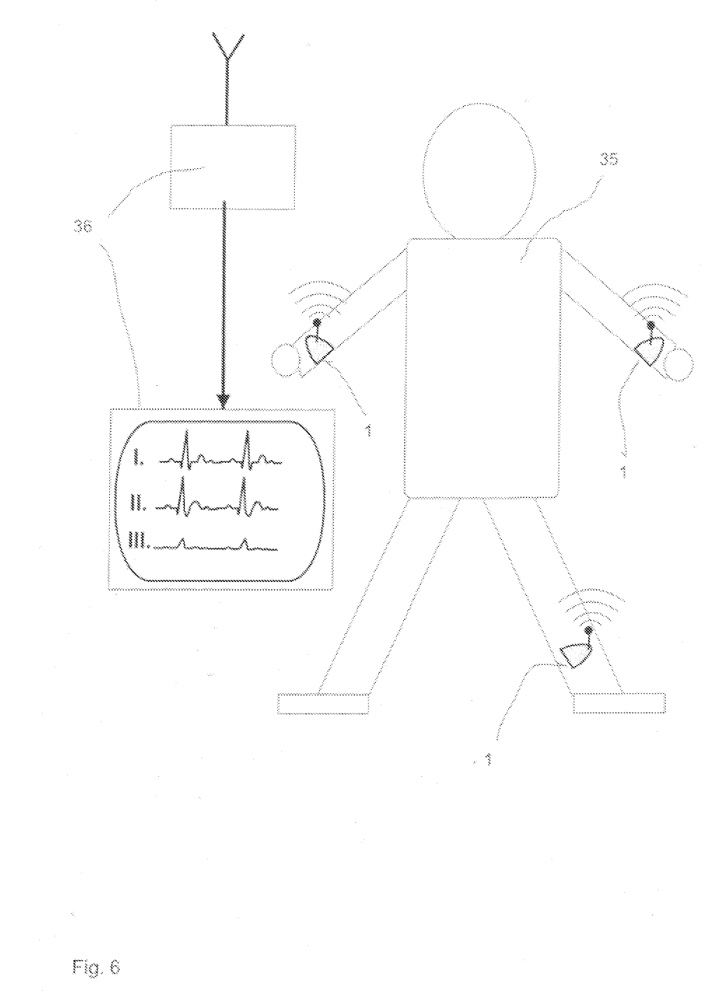

The potentials on the skin of a patient are determined at the electrodes 3, 4 and 5, and in this case, in which the electrodes 3, 4 and 5 are connected in a delta connection, three reference potentials are made available by the reference potential terminals 6, 7 and 8. By means of the respective signal amplifiers 15, 16 and 17, the differences between the potential of one of the electrodes 3, 4 and 5 and one of the reference potentials are measured, amplified and, relative to the ground potential of the battery 24, transmitted to the transmitting means, which comprises the multiplexing unit 20, the analog-digital converter 21, the controller 22 as well as the high-frequency transmission stage 23. After they have been digitized in the analog-digital converter 21, these individual signals can be transmitted to a receiving and evaluating unit in the digital form. Both bidirectional systems, for example, Bluetooth and Zigbee (at 2.45 GHz) and proprietary unidirectional systems (Bluerobin) may be used to transmit the digitized signals. As an alternative, it is also possible that the individual signals are first sent via a short-range radio connection to another electrode array, from which they are then transmitted to the receiving and evaluating unit. Both possibilities are described in detail with reference to FIGS. 6, 7 and 8.

Due to the fact that “virtual” reference signals, relative to which the individual signals are detected at the electrodes 3, 4 and 5, are made available at the reference potential terminals 6, 7 and 8, it is not necessary to connect the electrode arrays of a system for detecting an electrocardiogram to one another in order to make available a uniform reference potential. The electrode arrays 1 according to the present invention can rather be arranged independently from one another on the patient's body and they will then transmit the individual signals in a wireless manner to a receiving and evaluating unit, as this will be explained in more detail below. Since, moreover, a plurality of reference potentials are made available by the delta connection of the three electrodes 3, 4 and 5, a respective other reference potential can be used to detect one of the individual signals, so that the individual signals are detected independently from one another and a system comprising a plurality of electrode arrays 1 is less error-prone.

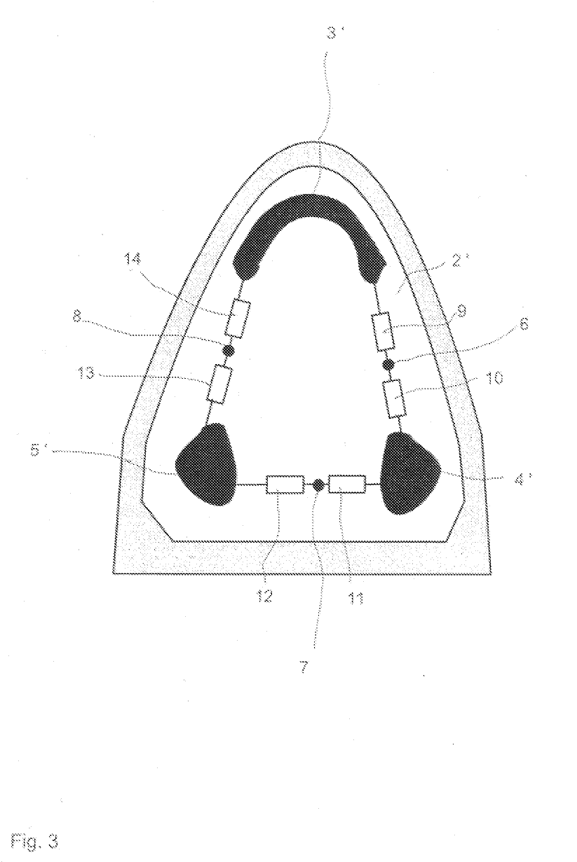

FIG. 3 shows an alternative embodiment of the lower part 2′ of the holding element, which can likewise be used in the first exemplary embodiment of an electrode array 1, and, unlike in FIG. 1, only the array of the first electrode 3′, of the second electrode 4′ and of the third electrode 5′ is shown together with the reference potential terminals 6, 7 and 8, while the signal amplifiers as well as the connection to the upper part are not shown.

Unlike in the alternative shown in FIG. 1 on the basis of commercially available circular electrodes, the electrodes 3′, 4′ and 5′ are not of a circular design here, but they extend over an extended area, which is semicircular in the case of the first electrode 3′, for better contact with the skin.

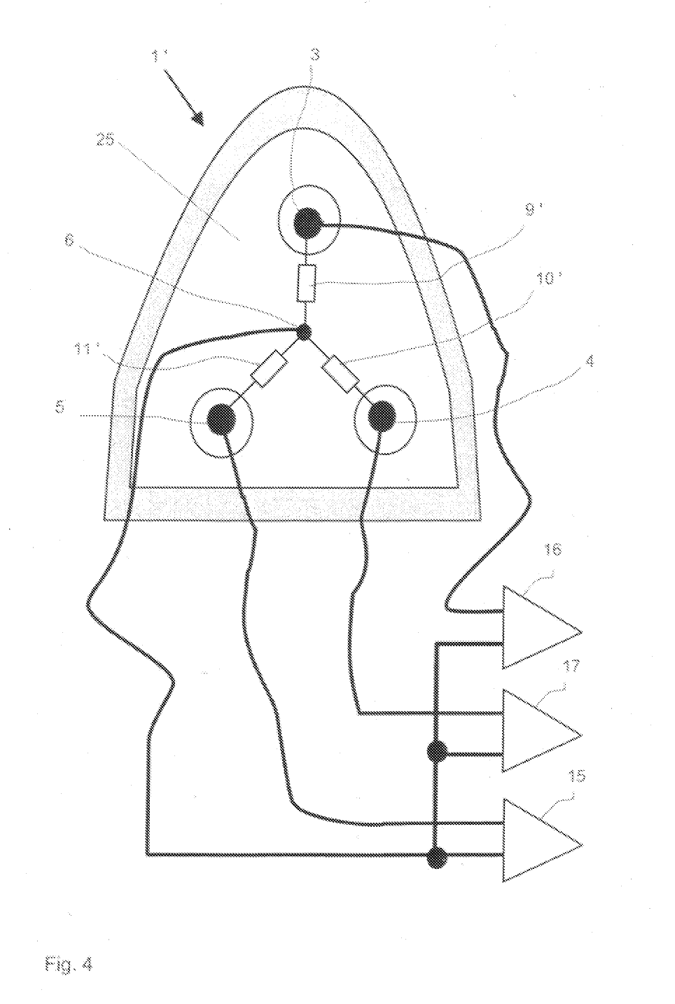

FIG. 4 shows a second exemplary embodiment of an electrode array 1′ according to the present invention, in which the electrode array 1′ likewise has a flat, sheet-like holding element 25, at which a first electrode 3, a second electrode 4 and a third electrode 5 are arranged, the electrodes 3, 4 and 5 being likewise fastened to the holding element 25 such that they are in contact with the skin of a patient when the holding element 25 is attached to the patient.

Furthermore, a reference potential terminal 6, which is connected to the electrodes 3, 4 and 5 in a star-shaped pattern via a first resistor 9′, a second resistor 10′ and a third resistor 11′ is provided at the holding element 25. The first reference potential terminal 6 therefore represents the “center” between the potentials of the electrodes 3, 4, 5.

As was already described in connection with the exemplary embodiment in FIGS. 1 and 2, the resistors 9′, 10′ and 11′ may be adjustable in the manner mentioned already in this case as well. In addition, it is conceivable that a capacitor (capacitance) and/or an inductor (inductance) each is connected between the reference potential terminal 6 and the electrodes 3, 4, 5 in order to embody a complex resistor.

Furthermore, the electrode array 1′ has a first signal amplifier 15, whose input is connected to the first reference potential terminal 6 and to the third electrode 5. This would already be sufficient for the detection according to the present invention of an individual signal by means of this electrode array 1′. However, since a plurality of individual signals shall be detected, a second signal amplifier 16 and a third signal amplifier 17, which are connected to the first electrode 3 and to the second electrode 4 in a manner analogous to the first signal amplifier 15, are provided in this preferred embodiment. Three individual signals can thus likewise be detected with this electrode array 1′, and they can then be digitized in the manner already explained in connection with FIGS. 1 through 3 and transmitted to a receiving and evaluating unit in a wireless manner.

As in the first exemplary embodiment, a “virtual” reference potential is made available at the reference potential terminal 6 in this case as well, and the individual signals at the electrodes 3, 4 and 5 are measured in relation to this by means of the signal amplifiers 15, 16 and 17, whose output signals are then related to the ground of a power supply unit, which is not shown here. Electrocardiogram signals can therefore also be detected with this electrode array 1′ without it being necessary to electrically connect the electrodes of the electrode array 1′ to one another to make available a common reference potential.

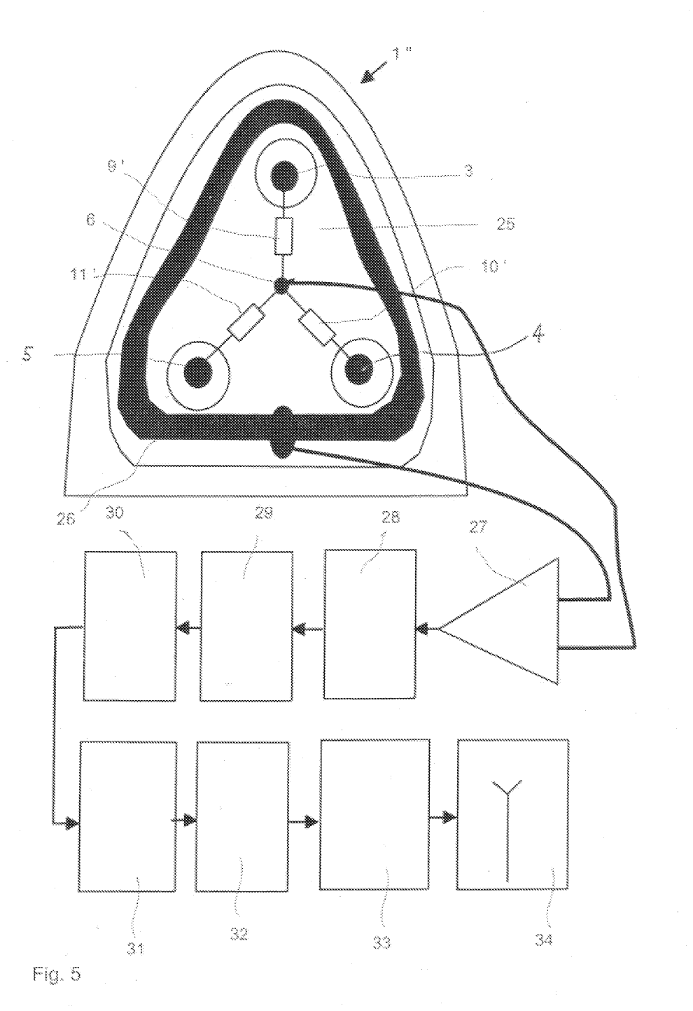

FIG. 5 shows a fourth exemplary embodiment of an electrode array 1″ according to the present invention, in which, just as in the second exemplary embodiment as well, the electrodes 3, 4 and 5 are connected in a star-shaped pattern to make available a reference potential at a reference potential terminal 6. Besides the three electrodes 3, 4 and 5, which are optionally provided with a ring consisting of contact gel, a fourth electrode 26 is also arranged at the holding element 25 for connection to the skin of a patient. The fourth electrode 26 surrounds the other electrodes 3, 4 and 5 in a ring-shaped pattern by means of a contact gel pad. In addition, the electrode array 1″ also has, besides the components already shown in FIG. 4, a fourth signal amplifier 27, whose input is connected to the first reference potential terminal 6 as well as to the fourth electrode 26. A fourth individual signal, which is likewise related to the ground of a power supply unit, which is not shown more specifically, can then be outputted at the output of the fourth signal amplifier 27. This signal can then be transmitted to a receiving and evaluating unit in a wireless manner. A signal filtering unit 28, an analog-digital converter 29, a coding unit 30, a modulation unit 31, a transmitting amplifier 32 and an antenna matching unit 33, which is connected to an antenna 34, are arranged for this purpose downstream of the fourth signal amplifier 27. The above-described common-mode components and asymmetries can be measured by means of the fourth electrode 26.

FIG. 6 shows a first embodiment of a system for detecting electrocardiogram signals according to the present invention. The system has electrode arrays 1, which may be designed according to one of the exemplary embodiments described above and have a holding element 25, a first electrode, a second electrode and a third electrode 3, 4, 5, respectively, and a transmitting means arranged at the holding element 25. The electrodes 3, 4, 5 are arranged at the holding element 25, and a first reference potential terminal 6, which is connected to the first electrode and the second electrode 3 and 4, respectively, and at which a reference potential is made available, is provided. The first reference potential terminal 6 and the third electrode 5 are connected to the transmitting means. The electrode arrays 1 are arranged on the right arm, the left arm and the left leg of a patient 35, the electrodes 3, 4, 5 of the electrode array 1 being in contact with the skin of the patient 35.

Since the electrode arrays 1 have three electrodes 3, 4 and 5 each, three individual signals each can be detected at the electrode arrays 1 and transmitted to a receiving and evaluating unit 36 in a wireless manner. The individual signals are designated by LA1, LA2, LA3, RA1, RA2, etc. The individual signals LA1, LA2, . . . are detected in the manner already described in connection with the electrode arrays 1, 1′ and 1″ without an electric connection being necessary between the individual electrode arrays 1, because one or more reference potentials are made available at each electrode array 1. In addition, the individual signals are digitized in the electrode arrays 1, so that digital signals are transmitted to the receiving and evaluating unit 36.

The necessary leads can be formed from the individual signals LA1, LA2, . . . in the receiving and evaluating unit 36 by complex difference formation (value and phase) as well as correction of signal distortions (asymmetry correction) by means of cross and autocorrelation methods) in order to make it possible to check the heart function of the patient 35.

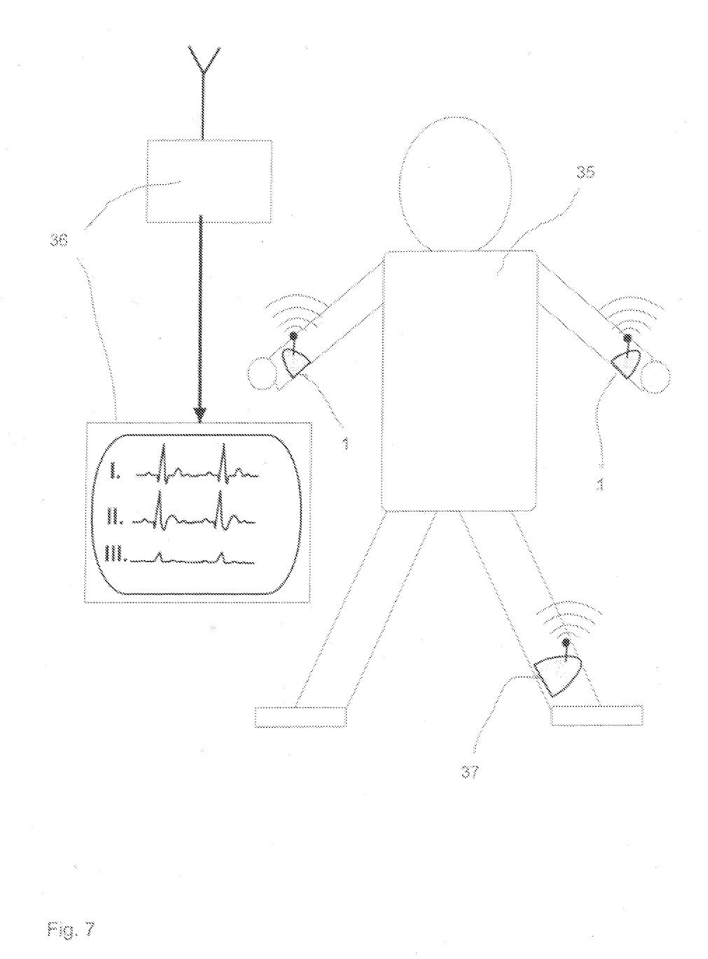

The second exemplary embodiment of a system according to the present invention, which is shown in FIG. 7, differs from that shown in FIG. 6 in that a transmission electrode array 37 is also provided on the left leg of the patient 35, besides the electrode arrays 1 on the left and right arms.

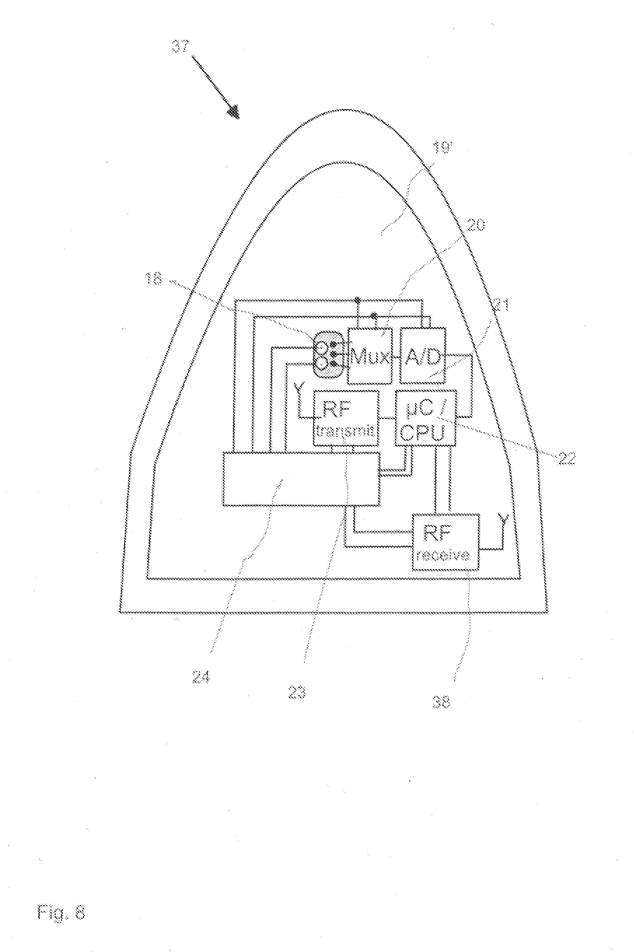

The lower part of the transmission electrode array 37 is designed as the lower part of an electrode array 1 shown in FIG. 1 and has especially a first electrode, a second electrode and a third electrode 3, 4 and 5, respectively, for connection to the skin of a patient 35 as well as to a reference potential terminal 6. The upper part 19′ of the transmission electrode array 37 is shown in FIG. 8, and this is likewise connected to the lower part, not shown, via a plug connection 18. Besides a battery 24, a multiplexing unit 20, an analog-digital converter 21, a controller 22 and a high-frequency transmission stage 23 are provided in the upper part 19′, these components form the transmitting means of the transmitting electrode array 37.

Furthermore, the upper part 19′ has a receiving unit 38, which is designed to receive signals of the transmitting means of the electrode arrays 1 of the system, and which are connected to the controller 22 of the transmitting unit.

A short-range radio connection is used to receive electrocardiogram signals in this embodiment in order to transmit the individual signals of the electrode arrays 1 on the left arm and the left leg to the receiving unit 38 of the transmission electrode array 37. Evaluation of the individual signals (formation of the leads) may optionally already take place at the transmission electrode array 37. The evaluated data can finally be transmitted to the receiving and evaluating unit 36 in a wireless manner in the manner already described (Bluetooth, Zigbee) in order to make it possible to visualize the data. However, it is also conceivable that the transmitting unit and the receiving and evaluating unit 36 are connected to one another by means of optical interfaces such as Infrared Detection and Acquisition (IrDA).

Thus, a staggered radio connection is embodied in this embodiment of the system between the electrode arrays 1, the transmitting electrode array 37 and the receiving and evaluating unit 36, which is associated with the advantage that the transmitting power to be provided at the electrode arrays 1 is low, because the signal must only be transmitted from these to the transmitting electrode array 37, which is likewise arranged on the patient's body.

Instead of a wireless connection between the transmitting unit and the receiving and evaluating unit 36, a cable connection is also conceivable as an alternative. The transmitting unit may now have a detachably connected transmitter, which is put on only when wireless transmission is intended.

The cable connection can thus be used in case of a hospitalized patient, whereas the wireless connection is used in case of a “mobile” patient.

Due to the system or the electrode arrays according to the present invention, it is no longer necessary for the electrode arrays 1, 37 arranged on the extremities of the patient 35 to be electrically connected to one another to make available a reference potential. This is rather generated at the electrode arrays 1, 37 themselves, so that disturbing cable connections between the electrode arrays 1, 37 can be eliminated. In addition, the accuracy of the calculated leads can be increased due to the plurality of detected and transmitted individual signals.

While specific embodiments of the invention have been shown and described in detail to illustrate the application of the principles of the invention, it will be understood that the invention may be embodied otherwise without departing from such principles.

What is claimed is:

1. An electrode array for a system for recording electrocardiogram signals, the electrode array comprising:
a sheet-like holding element;
a first electrode for connection to the skin of a patient, said first electrode being arranged at said holding element;
a second electrode for connection to the skin of a patient, said second electrode being arranged at said holding element;
a third electrode for connection to the skin of a patient, said third electrode being arranged at said holding element;
a transmitter means arranged at said holding element; and
a reference potential terminal connected to said first electrode and said second electrode and at which a reference potential is made available, said reference potential terminal and said third electrode being connected to said transmitter.

2. An electrode array in accordance with claim 1, wherein a first resistor is provided between said reference potential terminal and said first electrode and a second resistor is provided between said first reference potential terminal and said second electrode.

3. An electrode array in accordance with claim 2, wherein said first resistor and said second resistor are adjustable.

4. An electrode array in accordance with claim 1, wherein a first capacitor and/or a first inductor is connected between said reference potential terminal and said first electrode, and a second capacitor and/or a second inductor is connected between said reference potential terminal and said second electrode.

5. An electrode array in accordance with claim 1, wherein said reference potential terminal is connected to said third electrode and a resistor is provided between said first reference potential terminal and said third electrode.

6. An electrode array in accordance with claim 5, wherein said resistor is adjustable.

7. An electrode array in accordance with claim 5, wherein a capacitor and/or an inductor is connected between said reference potential terminal and said third electrode.

8. An electrode array in accordance with claim 5, wherein said first electrode and said second electrode are connected to the transmitter means.

9. An electrode array in accordance with claim 8, further comprising:
a first signal amplifier;
a second signal amplifier; and
a third signal amplifier wherein an input of said first signal amplifier is connected to said first reference potential terminal and to said third electrode, an input of said second signal amplifier is connected to said reference potential terminal and to said first electrode, an input of said third signal amplifier is connected to said first reference potential terminal and to said second electrode, and an output of said first signal amplifier, and output of said second signal amplifier and an output of said third signal amplifier are connected to said transmitter.

10. An electrode array in accordance with claim 1, wherein said reference potential terminal is a first reference potential terminal and further comprises:
a second reference potential terminal connected to said second electrode and connected to said third electrode; and
a third reference potential terminal connected to said first electrode and connected to said third electrode, said second reference potential terminal and said third reference potential terminal as well as said second electrode and said third electrode being connected to said transmitter.

11. An electrode array in accordance with claim 10, further comprising:
a first resistor provided between said first reference potential terminal and said first electrode;
a second resistor provided between said first reference potential terminal and said second electrode;
a third resistor provided between said second reference potential terminal and said second electrode;
a fourth resistor provided between said second reference potential terminal and said third electrode;
a fifth resistor provided between said third reference potential terminal and said third electrode; and
a sixth resistor provided between said third reference potential terminal and said first electrode.

12. An electrode array in accordance with claim 11, wherein each of said first resistor, said second resistor said third resistor, said fourth resistor, said fifth resistor and said sixth resistor are adjustable.

13. An electrode array in accordance with claim 10, further comprising:
a first capacitor and/or a first inductor connected between said first reference potential terminal and said first electrode;
a second capacitor and/or a second inductor connected between said first reference potential terminal and said second electrode;
a third capacitor and/or a third inductor connected between said second reference potential terminal and said second electrode;
a fourth capacitor and/or a fourth inductor connected between said second reference potential terminal and said third electrode; and
a fifth capacitor and/or a fifth inductor connected between said third reference potential terminal and said third electrode; and
a fifth capacitor and/or a fifth inductor connected between said third reference potential terminal and said first electrode.

14. An electrode array in accordance with claim 10, further comprising:
a first signal amplifier with an input connected to said first reference potential terminal and to said third electrode;
a second signal amplifier with an input connected to said second reference potential terminal and to said first electrode; and
a third signal amplifier with an input connected to said third reference potential terminal and to said second electrode, and an output of said first signal amplifier, and output of said second signal amplifier and an output of said third signal amplifier are connected to said transmitter.

15. An electrode array in accordance with claim 1, wherein a fourth electrode is provided and is attached to said holding element, said fourth electrode being connected to said transmitter.

16. An electrode array in accordance with claim 15, further comprising a signal amplifier, said fourth electrode and said reference potential terminal being connected to an input of said signal amplifier and an output of said signal amplifier is connected to said transmitter.

17. An electrode array in accordance with claim 15, wherein said fourth electrode has a ring-shape and surrounds said first, second and third electrodes.

18. An electrode array in accordance with claim 1, wherein:
said holding element has an upper part and a lower part, said electrodes (3, 4, 5) being fastened to said lower part; and
said transmitter is provided at said upper part.

19. An electrode array in accordance with claim 16, wherein:
said holding element includes a power supply part, and
said power supply part is provided with a power supply unit for supplying power to the electrode array.

20. An electrode array in accordance with claim 1, further comprising a receiving unit.

21. A system for recording electrocardiogram signals, the system comprising:
a receiving and evaluating unit; and
a plurality of electrode arrays, each of said electrode arrays comprising:
a sheet-like holding element;
a first electrode for connection to the skin of a patient, said first electrode being arranged at said holding element;
a second electrode for connection to the skin of a patient, said second electrode being arranged at said holding element;
a third electrode for connection to the skin of a patient, said third electrode being arranged at said holding element;
a transmitter arranged at said holding element;
a first reference potential terminal connected to said first electrode and connected to said second electrode, and at which a reference potential is made available, said reference potential terminal and said third electrode being connected to said transmitter.

22. A system in accordance with claim 21, further comprising:
a transmission electrode array comprising:
a sheet-like holding element;
a first electrode for connection to the skin of a patient, said first electrode being arranged at said holding element;
a second electrode for connection to the skin of a patient, said second electrode being arranged at said holding element;
a third electrode for connection to the skin of a patient, said third electrode being arranged at said holding element;
a transmitter arranged at said holding element;
a receiving unit arranged at said holding element;
a reference potential terminal connected to said first electrode and connected to said second electrode and at which a reference potential is made available, said reference potential terminal, said third electrode and the receiving unit being connected to the transmitting unit, said receiving unit receiving signals of said transmitter of said electrode arrays and said transmitter transmitting data to said receiving and evaluating unit.

23. A system in accordance with claim 22, wherein said transmitting unit is connected to said receiving and evaluating unit via a wireless connection.

24. A system in accordance with claim 23, wherein said transmitting unit has a transmitter detachably attached to said transmitting unit.

25. A system in accordance with claim 22, wherein said transmitting unit and said receiving and evaluating unit are connected to one another by means of optical interfaces.

26. A system in accordance with claim 22, wherein said transmitting unit is connected to said receiving and evaluating unit via a cable connection.
